(19) United States
(12) Patent Application Publication
(10) Pub. No.: US 2021/0307677 A1
(43) Pub. Date: Oct. 7, 2021

(54) SYSTEM FOR DETECTING EATING WITH SENSOR MOUNTED BY THE EAR

(71) Applicants: THE TRUSTEES OF DARTMOUTH COLLEGE, Hanover, NH (US); CLEMSON UNIVERSITY, Clemson, SC (US)

(72) Inventors: Shengjie Bi, White River Junction, VT (US); Tao Wang, White River Junction, VT (US); Nicole Tobias, Williamston, SC (US); Josephine Nordrum, Hanover, NH (US); Robert Halvorsen, Hanover, NH (US); Ron Peterson, Brattleboro, VT (US); Kelly Caine, Greenville, SC (US); Xing-Dong Yang, Hanover, NH (US); Kofi Odame, Hanover, NH (US); Ryan Halter, Lyme, NH (US); Jacob Sorber, Clemson, SC (US); David Kotz, Lyme, NH (US)

(21) Appl. No.: 17/265,032

(22) PCT Filed: Jul. 31, 2019
(86) PCT No.: PCT/US2019/044317

§ 371 Date: Feb. 1, 2021

Related U.S. Application Data
(60) Provisional application No. 62/712,255, filed on Jul. 31, 2018.

(51) Int. Cl. A61B 5/00; A61B 5/11; A61B 7/00

(52) U.S. Cl. CPC A61B 5/4542; A61B 5/6803; A61B 5/6898; A61B 2560/04; A61B 5/7264; A61B 5/725; A61B 7/006; A61B 5/1128

(57) ABSTRACT

A wearable device for detecting eating episodes uses a contact microphone to provide audio signals through an analog front end to an analog-to-digital converter to digitize the audio and provide digitized audio to a processor; and a processor configured with firmware in a memory to extract features from the digitized audio. A classifier determines eating episodes from the extracted features. In embodiments, messages describing the detected eating episodes are transmitted to a cell phone, insulin pump, or camera configured to record video of the wearer's mouth.

### PRIORITY CLAIM

The present application claims priority to U.S. Provisional Patent Application No. 62/712,255 filed Jul. 31, 2018, the entire content of which is hereby incorporated by reference.

### GOVERNMENT RIGHTS

This invention was made with government support under grant nos. CNS-1565268, CNS-1565269, CNS-1835974, and CNS-1835983 awarded by the National Science Foundation. The government has certain rights in the invention.

### BACKGROUND

Chronic disease afflicts many people; much of this disease is related to lifestyle, including diet, drinking, and exercise. Among medical and psychological conditions affected by diet where an accurate record of eating behaviors can be desirable, both for research and potentially for treatment, are anorexia nervosa, obesity, and diabetes mellitus. Psychological research also may make use of an accurate record of eating behaviors when studying such things as the effect of final exam stress on students—who often eat and snack while studying.

We define “eating” in this document as “an activity involving the chewing of food that is eventually swallowed.” This definition may exclude drinking actions, which usually do not involve chewing. On the other hand, consuming “liquid foods” that contain solid content (like vegetable soup) and require chewing is considered “eating”. Our definition also excludes chewing gum, since gum is not usually swallowed.

For the purposes of this document, we define an “eating episode” as: “a period of time beginning and ending with eating activity, with no internal long gaps, but separated from each adjacent eating episode by a gap greater than 15 minutes, where a ‘gap’ is a period in which no eating activity occurs.”

### SUMMARY

We have devised a head-mounted eating monitor adapted to detect episodes of eating and transmit data regarding such episodes over a short-range digital radio.

In an embodiment, a device adapted to detect eating episodes includes a contact microphone coupled to provide audio signals through an analog front end; an analog-to-digital converter configured to digitize the audio signals and provide digitized audio to a processor; and a processor configured with firmware in a memory to extract features from the digitized audio, and the firmware including a classifier adapted to determine eating episodes from the extracted features. In particular embodiments, the device includes a digital radio, the processor configured to transmit information comprising time and duration of detected eating episodes over the digital radio. In particular embodiments, the device includes an analog wake-up circuit configured to arouse the processor from a low-power sleep state upon the audio signals being above a threshold.

In embodiments, a system designated includes a camera, the camera configured to receive detected eating episode information over a digital radio from a device adapted to detect eating episodes including a contact microphone coupled to provide audio signals through an analog front end; an analog-to-digital converter configured to digitize the audio signals and provide digitized audio to a processor; and a processor configured with firmware in a memory to extract features from the digitized audio, and a classifier adapted to determine eating episodes from the extracted features. The camera is further adapted to record video using the camera upon receipt of detected eating episode information.

In another embodiment, a system includes an insulin pump, the insulin pump configured to receive detected eating episode information over a digital radio from a device adapted to detect eating episodes including a contact microphone coupled to provide audio signals through an analog front end; an analog-to-digital converter configured to digitize the audio signals and provide digitized audio to a processor; and a processor configured with firmware in a memory to extract features from the digitized audio, and a classifier adapted to determine eating episodes from the extracted features. The insulin pump is further adapted to request user entry of meal data upon receipt of detected eating episode information.

### BRIEF DESCRIPTION OF THE DRAWINGS

FIG. 1A is an illustration of where the contact microphone is positioned against skin over a tip of a mastoid bone.

FIG. 1B is a block diagram of a system incorporating the monitor device of FIG. 1C for detecting episodes of eating.

FIG. 1C is a block diagram of a monitor device for detecting episodes of eating.

FIGS. 2A, 2B, 2C, and 2D are photographs of a particular embodiment illustrating a mechanical housing attachable to human auricles showing location of the microphone.

FIG. 3 is a photograph of an embodiment mounted in a headband.

FIG. 4 is a schematic diagram of a wake-up circuit that permits partial shutdown of the monitor device when the contact microphone is not receiving significant signals.

FIG. 5 is a flowchart illustrating how features are determined for detecting eating episodes.

### DETAILED DESCRIPTION OF THE EMBODIMENTS

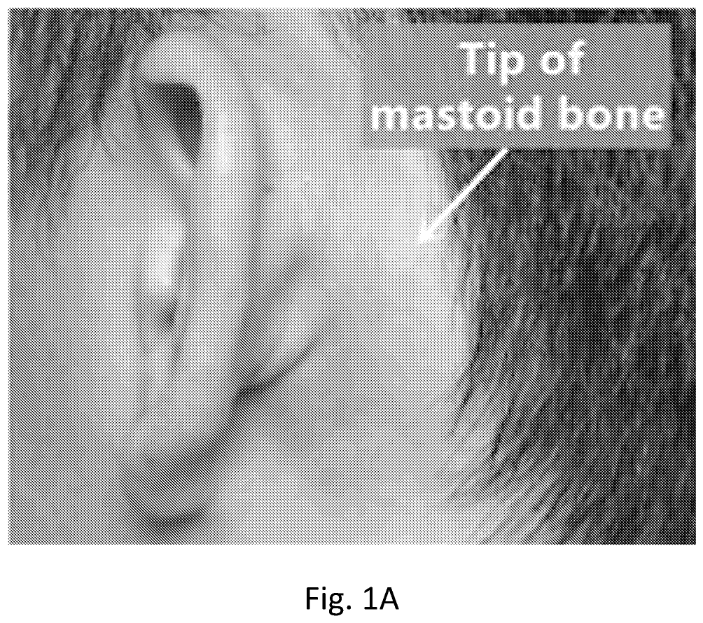

Our device 100 (FIG. 1C) includes within a compact, wearable housing a contact microphone 102 and analog front end 103 (AFE) for signal amplification, filtering, and buffering, together with a battery 104 power system that may or may not include a battery-charging circuit. The device 100 also includes a microcontroller processor 106 configured by firmware 110 in memory 108 to perform signal sampling and processing, feature extraction, eating-activity classification, and system control functions. The processor 106 is coupled to a digital radio 112 that in an embodiment is a Bluetooth low energy (BLE)-compliant radio and a “flash” electrically erasable and electrically writeable read-only memory that in an embodiment comprises a micro-SD card socket configured for data storage of records of eating events. The signal and data pipeline from the contact microphone includes AFE-based signal shaping, microcontroller processor-based analog-to-digital conversion, and within processor 106 as configured by firmware 110 in memory 108 on-board feature extraction and classification, and data transmission and storage functions. The processor 106 is also coupled to a clock/timer device 116 that allows accurate determination of eating episode time and duration.

A system 160 (FIG. 1B) incorporates the eating monitor 100 (FIG. 1C), 162 (FIG. 1B). In embodiments, the eating monitor 162 is configured to use digital radio 112 to transmit time and duration of eating episodes to cell phone 164 or other body-area network hub, where an appropriate application (app) records each occurrence of an eating episode in a database 166 and may use a cellular internet connection to transmit eating episodes over the internet (not shown) to a server 168 and enter those episodes into a database 170. In some embodiments, either the cell phone 164 or other body-area network hub relays detected eating episodes to a cap 171-mounted camera 172 or to an insulin pump 174; in some embodiments, both a cap-mounted camera and an insulin pump may be present.

In some embodiments, the cap-mounted camera 172 is configured to record video of a patient's mouth to provide information on what and how much was eaten during each detected eating episode, each video recording begins at a first time window when eating is detected by eating monitor 162, and extends to a time window after eating is no longer detected. In some embodiments, the insulin pump is prompted to beep, requesting user entry of meal data, whereupon insulin dosage may be adjusted according to the amount and caloric content of food eaten according to the meal data.

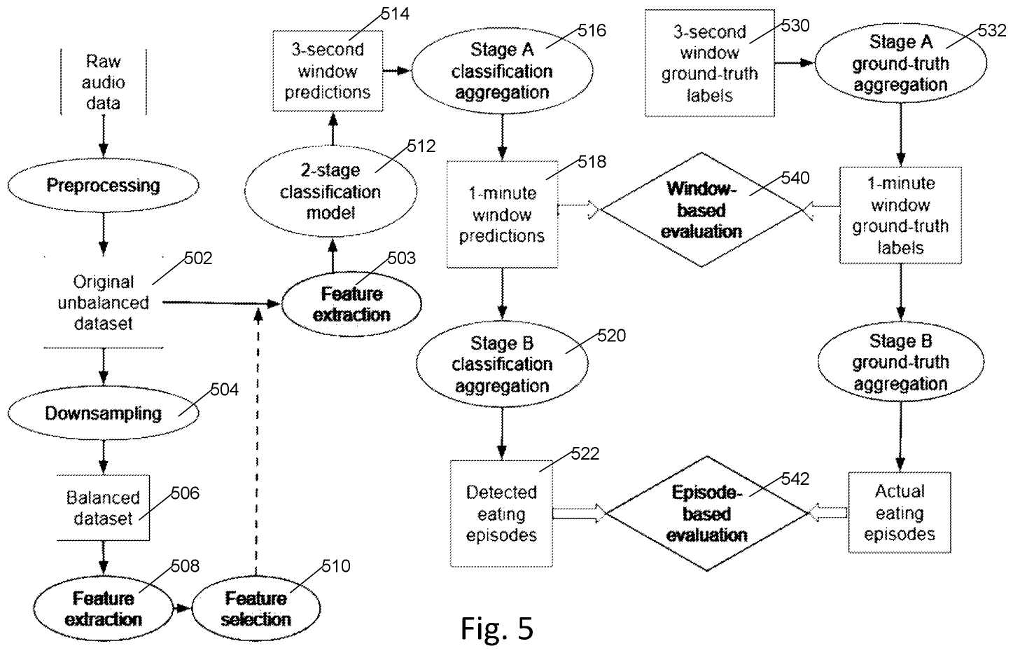

In preparing and testing our classifier, we derived a field data set of data with 3-second time windows labeled as eating and non-eating for use as a feature determination and training set. Windows were labeled as eating or non-eating based upon video recorded by a “ground truth” detector including a hat-mounted camera configured to film mouths of human subjects. In our original field data set, the number of windows labeled as non-eating was significantly larger than the ones labeled as eating (the time-length ratio of data labeled as non-eating and eating is 6.92:1). When we selected features on this dataset, the top features returned provide us relatively good accuracy, but not always good recall and precision. However, recall and precision may be important metrics for some eating-behavior studies, so we first converted the original unbalanced dataset 502 (FIG. 5) to a balanced dataset by randomly down-sampling 504 the number of non-eating windows so that we had equal numbers of non-eating windows and eating windows in a balanced dataset 506. We then performed feature extraction 508 and selection on the balanced dataset (See FIG. 5).

For each time window, we used the open-source Python package tsfresh2 to extract a common set of 62 categories of feature from both time and frequency domains. Each feature category in this set can consist of up to hundreds of features when the parameters of the feature category vary. In our case, we extracted more than 700 features in total. We then selected relevant features based on feature significance scores and the Benjamin-Yekutieli procedure. We evaluated each feature individually and independently with respect to its significance in detecting eating, and generated a p-value to quantify its significance. Then, the Benjamini-Yekutieli procedure evaluated the p-value of all features to determine which features to keep for use in the eating monitor. After removing irrelevant features, considering the limited computational resources of wearable platforms, we further selected a smaller set of features using the Recursive Feature Elimination (RFE) algorithm with a Lasso kernel (5<k<60).

Table 1 summarizes the top 40 features.

TABLE 1
Top 40 features selected by RFE algorithm

| | | No. |
|---|---|---|
| Feature Category | Description | Features |
| Coefficients of 1D | DFT coefficients | 29 |
| discrete Fourier | | |
| transform (DFT) | | |
| Range count | Count of pulse-code-modulated (PCM) | 1 |
| | values within a specific range | |
| Value count | Count of occurrences of a PCM value | 1 |
| Number of crossings | Count of crossings of a specific value | 3 |
| Sum of reoccurring | Sum of all values that present more than | 1 |
| values | once | |
| Sum of reoccurring | Sum of all data points that present more | 1 |
| data points | than once | |
| Count above mean | Number of values that are higher than | 1 |
| | mean | |
| Longest strike | Length of the longest consecutive | 1 |
| above mean | subsequence > mean | |
| Number of peaks | Number of peaks at different width scales | 2 |

Finally, we then extracted the same k features from the original unbalanced dataset to run the classification experiments (5<k<60).

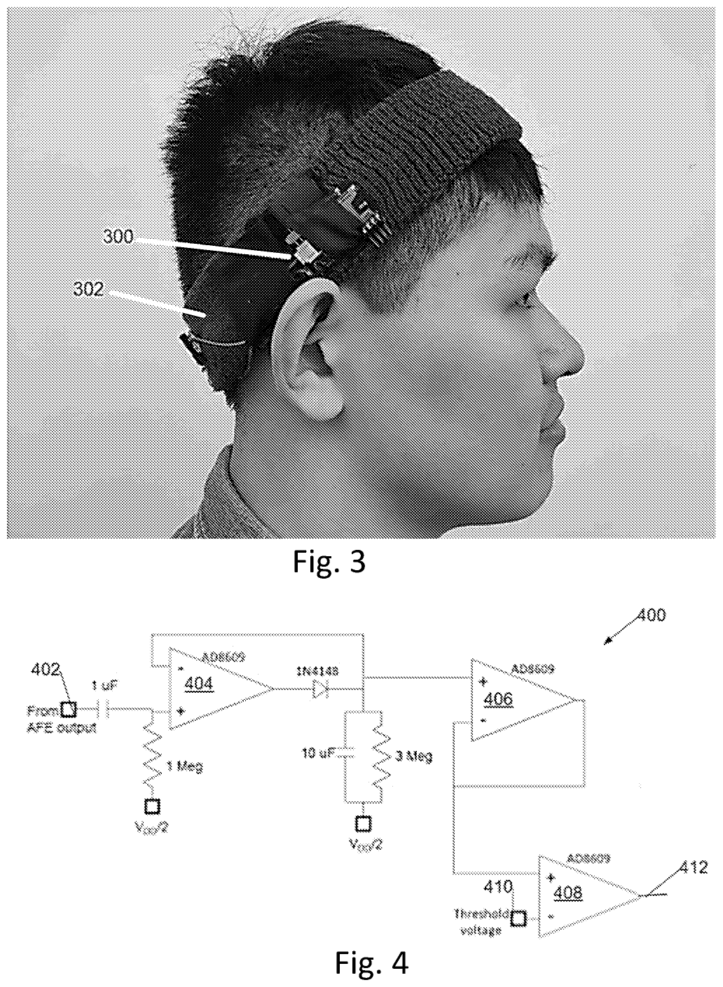

We designed a two-stage classifier 512 to perform a binary classification on the original unbalanced dataset, using the set of features selected above. In Stage I, we used simple thresholding to filter out the time windows that seemed to include silence; in production systems, Stage 1 of the classifier is replaced with the analog-based wake-up circuit of FIG. 4. We calculated the threshold for Stage 1, or the wake-up circuit, by averaging the variance of audio data across multiple silent time windows. We collected this silent data during a preliminary controlled data-collection session. We identified time windows in the field data that had lower variance than the pre-calculated threshold and marked them as “evident silence periods”. During testing, we labeled the time windows in the testing set that were evident silence periods as “non-eating”. After separating training and testing data, we trained our stage II classifier on the training set excluding the evident silence periods or intervals.

In stand-alone embodiments, the wake-up circuit discussed with reference to FIG. 4 serves to detect silent intervals; these silent intervals are presumed to be non-eating time windows without performing stage II of the classifier. As running stage II of the classifier is unnecessary on silent intervals, the processor is permitted to shut itself down until the wake-up circuit detects a non-silent interval or another event—such as a timer expiration or digital radio packet reception—requires processor attention.

In an embodiment, Stage II of the classifier 512 is a Logistic Regression (LR) classifier with weights as appropriate for each feature determined to be significant. Weights are determined using the open source Python package scikit-learn to train the LR classifier; this package is available at scikit-learn.org. In alternative embodiments, we have experimented with Gradient Boosting, Random Forest, K-Nearest-Neighbors (KNN), and Decision Tree classifiers. Since many eating episodes last far longer than three seconds, we have also used rolling one-minute windows with 50% overlap, each one-minute window including twenty of the three-second intervals, classifying each one-minute window as eating if more than two of the three-second intervals within it are classified as eating, and determine eating episodes as a continuous group of one-minute windows that are classified as eating.

Training required labeling 3-second time windows of training set audio by using a ground truth detector, the ground truth detector being a camera positioned on a cap to view a subject's mouth. Labeled 3-second time windows were similarly aggregated 532 into one-minute eating windows.

The stand-alone embodiments are similar, they extract features from three second time windows of digitized audio, the features being those determined as significant using the feature determination and training set, and the stage II classifier used in these embodiments uses the extracted features, as trained on the feature determination and training set, to determine windows including eating episodes. The net effect of the feature extraction and classification is to determine which of 3-second time intervals of pulse-code-modulated (PCM) audio represent eating activity 514, and which intervals do not represent eating activity, and then determines 516 which of the one-minute rolling time windows represent eating and which do not. One-minute time windows determined to include eating activity are then aggregated 518 into “eating episodes” 520, for which time and duration are recorded as eating episode data.

Running the training set of laboratory sound data through the feature extractor and classifier of a stand-alone embodiment, using the features determined as significant and weights as determined above, gives detection results as listed in Table 2 for the three-second intervals.

TABLE 2
Stage II Classifier Performance

| | | | | Weighted | F1 |
|---|---|---|---|---|---|
| Classifier | Accuracy | Precision | Recall | Accuracy | Score |
| Logistic Regression | .928 | .757 | .808 | .879 | .775 |
| Gradient Boosting | .924 | .769 | .757 | .856 | .751 |
| Random Forest | .891 | .629 | .866 | .881 | .718 |
| K Nearest | .888 | .629 | .810 | .858 | .689 |
| Neighbors | | | | | |
| Decision Trees | .753 | .394 | .914 | .819 | .539 |

We place the contact microphone behind the ear, directly over the tip of mastoid bone (FIG. 1A); this location has been shown to give a strong chewing signal to a contact microphone. In a prototype, the contact microphone is a CM-01B from Measurement Specialties. This microphone uses a polyvinylidene fluoride (PVDF) piezoelectric film combined with a low-noise electronic preamplifier to pick up sound applied to a central rubber pad, and a metal shell minimizes external acoustic noise. The 3 dB bandwidth of the microphone ranges from 8 Hz to 2200 Hz. Signals from the microphone pass to the AFE 103 where it is amplified and bandlimited to a 0-250 Hz frequency range before being sampled and digitized into PCM signals at 500 samples per second by ADC 105; a three-second window of samples is stored for analysis by processor 106.

To conserve power, we use a low-power wake-up circuit 118, 400 (FIG. 4) to determine when the AFE is receiving audio signals exceeding a preset threshold. Signals 402 from the AFE are passed into a first op-amp 404 configured as a peak detector with a long decay time constant, then the detected peaks are buffered in a second op-amp 406 and compared in a third op-amp 408 to a predetermined threshold 410 to provide a wake-up signal 412 to the processor 106 (FIG. 1). When the wake-up circuit detects sound, it triggers the processor to switch from sleep state to wake-up state and begin sampling, processing, and recording data from the microphone.

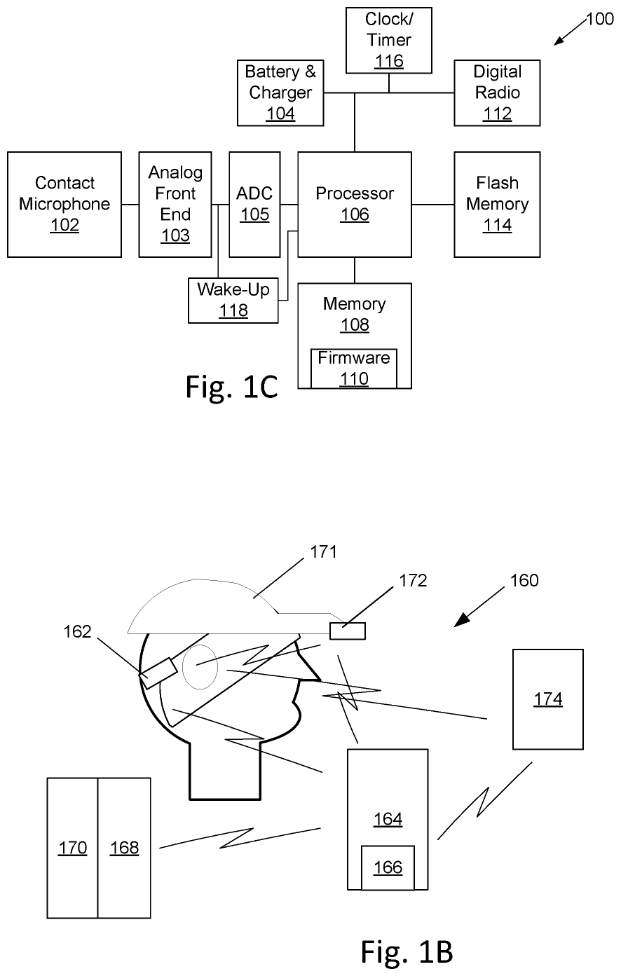

An embodiment 200 includes a 3D-printed ABS plastic frame that wraps around the back of a wearer's head and houses a printed circuit board (PCB) bearing the processor, memory, and battery, and the contact microphone (FIG. 2A-2D). Soft foam supports the frame as it sits above a wearer's ears. There are grooves in the enclosure making the device compatible with wear of most types of eyeglasses. The contact microphone is adjustable, backed with foam that can be custom fit to provide adequate contact on different head shapes while providing proper contact of the microphone with skin over the mastoid bone. An adjustable microphone ensures that the device can be adapted to several head shapes and bone positions.

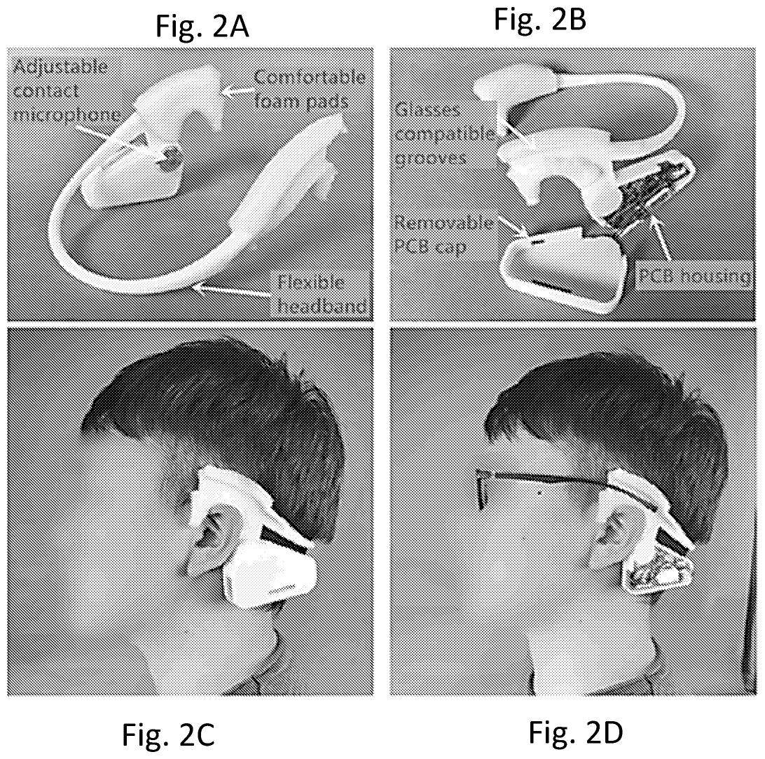

An alternative embodiment 300 (FIG. 3) is integrated into an elastic headband 302, so it can be worn like a hairband or sweatband. This embodiment is flexible (literally) and thus fits heads of multiple different sizes and shapes without adjustment, better than the embodiment of FIGS. 2A-2D. It does a good job of keeping the microphone pressed against the skin over the mastoid bone.

### Validation Experiments

We collected field data with 14 participants for 32 hours in free-living conditions and additional eating data with 10 participants for 2 hours in a laboratory setting. We fused an off-the-shelf wearable miniature camera mounted under the brim of a baseball cap to record video during the field studies as a ground truth detector, and three-second time windows of PCM audio were labeled as eating or non-eating accordingly. The camera was directed at the mouth of the participants. One-minute intervals aggregated from the classifier were compared 540 to one-minute intervals aggregated from the ground truth labels. One-minute intervals with ground-truth labels were aggregated into eating episodes similarly to one minute intervals aggregated from classifier three-second windows and compared 542 to the one minute intervals aggregated from classifier data.

During laboratory studies, we asked participants to eat six different types of food, one after the other. The food items included three crunchy types (protein bars, baby carrots, crackers) and three soft types (canned fruits, instant foods, yogurts). We asked the participants to chew and swallow each type of food for two minutes. During this eating period, participants were asked to refrain from performing any other activity and to minimize the gaps between each mouthful. After every 2 minutes of eating an item, participants took a 1-minute break so that they could stop chewing gradually and prepare for eating another type of food.

A field study using a prototype device and a hat-visor-mounted video camera for ground truth detection achieved accuracy exceeding 92.8% and F1 score exceeding 77.5% for eating detection. Moreover, our device successfully detected 20-24 eating episodes (depending on the metrics) out of 26 in free-living conditions. We demonstrate that our device could sense, process, and classify audio data in real time.

We focus on detecting eating episodes rather than sensing generic non-speech body sound.

As we define eating as “an activity involving the chewing of food that is eventually swallowed,” a limitation is that our system relies on chewing detection. If a participant performed an activity with a significant amount of chewing but no swallowing (e.g., chewing gum), our system may output false positives; activities with swallowing but no chewing (e.g., drinking) will not be detected as eating although they may be of interest to some dietary studies. More explorations in swallowing recognition can help overcome this limitation.

Stand-alone eating monitors record 502 three-second time windows of audio, extract features therefrom 503, classify 512 the windows based on the extracted features, aggregate 516 classified windows into rolling one-minute windows, and aggregate 520 the one-minute windows into eating episodes into detected eating episodes 522 as shown on FIG. 5, but omit ground-truth labeling, aggregation, and comparison.

Combinations

The devices, methods, and systems herein disclosed may appear in multiple variations and combinations. Among combinations specifically anticipated by the inventors are:

A device designated A adapted to detect eating episodes including a contact microphone coupled to provide audio signals through an analog front end; an analog-to-digital converter configured to digitize the audio signals and provide digitized audio to a processor; and a processor configured with firmware in a memory to extract features from the digitized audio, and a classifier adapted to determine eating episodes from the extracted features.

A device designated AA including the device designated A further including a digital radio, the processor configured to transmit information comprising time and duration of detected eating episodes over the digital radio.

A device designated AB including the device designated A or AA further including an analog wake-up circuit configured to arouse the processor from a low-power sleep state upon the audio signals being above a threshold.

A device designated AC including the device designated A, AA, or AB wherein the classifier includes a classifier configured according to a training set of digitized audio windows determined to be eating and non-eating time windows having audio that exceeds a threshold.

A device designated AD including the device designated A, AA, AB, or AC wherein the classifier is selected from the group of classifiers consisting of Logistic Regression, Gradient Boosting, Random Forest, K-Nearest-Neighbors (KNN), and Decision Tree classifiers.

A device designated AE including the device designated AD wherein the classifier is a logistic regression classifier.

A system designated B including a camera, the camera configured to receive detected eating episode information over a digital radio from the device designated AA, AB, AC, AD, or AE, and to record video upon receipt of detected eating episode information.

A system designated C including an insulin pump, the insulin pump configured to receive detected eating episode information over a digital radio from the device designated AA, AB, AC, AD, or AE, and to request user entry of meal data upon receipt of detected eating episode information.

A method designated D of detecting eating includes: using a contact microphone positioned over the mastoid of a subject to receive audio signals from the subject; determining if the audio signals exceed a threshold; and, if the audio signals exceed the threshold, extracting features from the audio signals, and using a classifier on the features to determine eating episodes.

A method designated DA including the method designated D and further including using an analog wake-up circuit configured to arouse a processor from a low-power sleep state upon the audio signals being above a threshold.

A method designated DB including the method designated DA wherein the classifier includes a classifier configured according to a training set of digitized audio determined to be eating and non-eating time windows that exceed a threshold.

A method designated DC including the method designated D, DA, or DB wherein the classifier is selected from the group of classifiers consisting of Logistic Regression, Gradient Boosting, Random Forest, K-Nearest-Neighbors (KNN), and Decision Tree classifiers.

A method designated DE including the method designated DD wherein the classifier is a logistic regression classifier.

A device designated AF including the device designated A, AA, AB, AC, AD, or AE, or the system designated B or C, wherein the features are determined according to a recursive feature elimination algorithm.

Changes may be made in the above system, methods or device without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

1. A device adapted to detect eating episodes comprising:
a contact microphone coupled to provide audio signals through an analog front end;
an analog-to-digital converter configured to digitize the audio signals and provide digitized audio to a processor; and
a processor configured with firmware in a memory to extract features from the digitized audio, the firmware comprising a classifier adapted to determine eating episodes from the extracted features.

2. The device of claim 1 further comprising a digital radio, the processor configured to transmit information comprising time and duration of detected eating episodes over the digital radio.

3. A device of claim 1 further comprising an analog wake-up circuit configured to arouse the processor from a low-power sleep state upon the audio signals being above a threshold.

4. A device of claim 2 wherein the classifier includes a classifier configured according to a training set of digitized audio time windows determined to be eating and non-eating time windows, the digitized audio time windows of the training set having audio that exceeds a threshold.

5. A device of claim 3 wherein the classifier is selected from the group of classifiers consisting of Logistic Regression, Gradient Boosting, Random Forest, K-Nearest-Neighbors (KNN), and Decision Tree classifiers.

6. The device of claim 5 wherein the classifier is a logistic regression classifier.

7. A system comprising a camera, the camera configured to receive detected eating episode information over a digital radio from the device of claim 4, and to record video upon receipt of detected eating episode information.

8. A system comprising an insulin pump, the insulin pump configured to receive detected eating episode information over a digital radio from the device of claim 3, and to request user entry of meal data upon receipt of detected eating episode information.

9. A method of detecting eating comprising:
using a contact microphone positioned over the mastoid of a subject to receive audio signals from the subject;
determining whether the audio signals exceed a threshold; and
if the audio signals exceed the threshold,
extracting features from the audio signals, and
using a classifier on the features to determine periods where the subject is eating.

10. The method of claim 9 further comprising using an analog wake-up circuit configured to arouse a processor from a low-power sleep state upon the audio signals being above a threshold.

11. The method claim 9 wherein the classifier includes a classifier configured according to a training set of digitized audio windows determined to be eating and non-eating time windows having audio that exceeds a predetermined threshold.

12. The method of claim 10, wherein the classifier is selected from the group of classifiers consisting of Logistic Regression, Gradient Boosting, Random Forest, K-Nearest-Neighbors (KNN), and Decision Tree classifiers.

13. The method of claim 12 wherein the classifier is a logistic regression classifier.
